(19) United States
(12) Patent Application Publication
(10) Pub. No.: US 2024/0000537 A1
(43) Pub. Date: Jan. 4, 2024

(54) APPARATUS FOR MARKING AN ENTRY POINT ON THE SKIN OF A PATIENT FOR A NEEDLE BIOPSY SUPERVISED BY MAGNETIC RESONANCE

(71) Applicant: Siemens Healthcare GmbH, Erlangen (DE)

(72) Inventors: Alexander Sebastian Brockmann, Bidingen (DE); Florian Maier, Buckenhof (DE)

(21) Appl. No.: 18/205,703

(22) Filed: Jun. 5, 2023

(30) Foreign Application Priority Data
Jun. 30, 2022 (DE) 10 2022 206 668.5

(51) Int. Cl. A61B 90/00; A61B 90/11

(52) U.S. Cl. CPC A61B 90/39; A61B 90/11; A61B 2090/3933; A61B 2090/3987; A61B 2090/3954

(57) ABSTRACT

A marking apparatus for applying an optically visible liquid and/or a liquid visible to MR to a patient in a magnetic resonance tomograph. The marking apparatus has a longitudinal body made of material visible to MR and a delivery opening at an end of the longitudinal body.

### CROSS REFERENCE TO RELATED APPLICATIONS

This application claims the benefit of DE 102022206668.5 filed on Jun. 30, 2022, which is hereby incorporated by reference in its entirety.

### FIELD

Embodiments relate to a marking apparatus for marking an entry position for an injection apparatus as part of interventional MR imaging.

### BACKGROUND

In minimally invasive medical procedures medical instruments, for example catheters and/or intervention needles, are introduced into a patient. The procedure is usually supervised using imaging. Imaging supervision makes it possible to record images in which the medical instrument is made visible with regard to its anatomical surroundings. While x-ray imaging has classically been used for imaging supervision of minimally invasive medical interventions, especially fluoroscopy, it has since also been proposed that magnetic resonance devices, thus magnetic resonance imaging (MR imaging), be used for imaging supervision. This is typically referred to as interventional MR imaging. During use of so-called closed magnetic resonance devices, which have a main magnet with a cylindrical patient receiving area, in which the homogeneity volume is located, work has to be carried out in extremely confined spaces in such cases, so that any support of the person carrying out the intervention is useful.

A specific type of medical instrument that are frequently employed for minimally invasive medical interventions are intervention needles, that are used for biopsy, ablation or brachytherapy for example. It has also already been proposed with regard to intervention needles that the intervention needles be propagated using real-time magnetic resonance control. To do this it is necessary for the intervention needle to be placed to be introduced at an entry position defined in a planning step and be introduced with a specific entry angle, in order then to be pushed forward along the defined trajectory to a target position, for example to a lesion.

In the planning phase in this case the anatomical boundary conditions and the technical restrictions that are produced by the positioning of the patient in the patient receiving area are taken into account. Anatomical boundary conditions relate in this case for example to the localization not only of the lesion but also of bones, vessels, and other structures to be protected. The planning, for example the definition of the entry position, the trajectory, and the target position, may for example be carried out on MR imaging data recorded with the magnetic resonance device, for example three-dimensional MR image data.

For carrying out the minimally invasive intervention in an efficiently timed manner and one that is as protective and pain-free as possible for the patient, it is vital that the entry position and where necessary also the entry angle in accordance with the planning may be found without subsequent repositioning.

Approaches have already been proposed for realizing this for a magnetic resonance device. A manual marking and representation of the entry position and the entry angle by the finger of a person carrying out the intervention is possible. For this the finger of the radiologist is positioned under imaging supervision, thus realtime MR imaging, at the entry position in such a way in which the intervention needle is to be introduced later. However due to the large diameter of the finger compared to the diameter of the intervention needle, accurate planning is hardly possible in this way. The entry position identified in this way is subsequently marked on the skin of the patient with the aid of a pen and/or a marker visible in MR imaging data. Likewise the fixing of a grid visible in MR data to the surface of the patient is known and the entry position is determined relative to the grid, for example by numbers of grid lines. In another approach it was proposed that the location of the entry position in the longitudinal direction of the patient receiving area be marked by a laser point or generally a light pattern projected on the surface of the patient. Even after planning and fixing the entry position with the aid of MR image data these approaches only allow an imprecise marking. This means that the entry position for the medical instrument used in a minimally invasive medical intervention may deviate from the fixed entry position, whereby the risk and the duration of the minimally invasive medical intervention increase.

From publication DE 10 2021 205 811 (not yet published) an apparatus for marking an entry position is known.

### BRIEF SUMMARY AND DESCRIPTION

The scope of the present disclosure is defined solely by the claims and is not affected to any degree by the statements within this summary. The present embodiments may obviate one or more of the drawbacks or limitations in the related art.

Embodiments provide improved support for a person carrying out a minimally invasive medical intervention supported by images from MR imaging.

The marking apparatus for applying an optically and/or MR visible liquid to a patient in a magnetic resonance tomograph includes a longitudinal body with material visible to MR. In this case a material that is referred to as being visible to MR is one that is shown in an imaging of the patient with the magnetic resonance tomograph. The material visible to MR may for example be water or a gel containing water or another substance with a Larmor frequency close to the Larmor frequency of the nuclei to be detected in the patient with the magnetic resonance tomograph. It is also conceivable for the material to be visible from artifacts, such as for example through a magnetic susceptibility in the imaging. The body of the marking apparatus may consist entirely or in part of the material visible to MR or may contain said material in hollow spaces.

The body in this case is not necessarily an outer shell of the marking apparatus but may also relate to a structure extending along the marking apparatus, of which the position or alignment may be deduced from an alignment of the marking apparatus. A delivery opening for the liquid is arranged at an end of the longitudinal body.

The marking apparatus includes an actuation element that is arranged laterally on the longitudinal body of the marking apparatus.

The actuation element, when actuated by the finger, controls a delivery of the liquid at the delivery opening. For example, by pressing on the actuation element in the direction of the body or a movement in the longitudinal direction or tangentially or circumferentially around the body a valve for delivering the liquid is opened or a delivery apparatus for the liquid is actuated.

In an advantageous way the lateral arrangement of the actuation element allows an actuation without changing the position of the guiding hand and the marking apparatus.

In an embodiment of the marking apparatus the marking apparatus includes a liquid conductor, that is arranged movably in the delivery opening. A liquid conductor may for example be a closed tube or small tube. A fluid conductor may also be used that is based on capillary forces, for example a bundle of fibers or a channel between parallel delimitations.

An axial guide may be provided for the liquid conductor. The axial guide allows a movement of the liquid conductor in a direction that is parallel to an axis of the largest extent of the marking apparatus or that encloses an angle of less than 5 degrees, 10 degrees or 20 degrees with it. The liquid conductor is movable on the axial guide by the actuation element from a delivery position in which the liquid conductor delivers the liquid at the delivery opening to a surface of a patient located there, into a standby position, in which there cannot be any delivery of the liquid to a surface of a patient located at the delivery opening. In the delivery position the liquid conductor may project up to or even beyond the delivery opening of the marking apparatus, so that it may touch the patient, and in the standby position is withdrawn into an inside of the marking apparatus so that the liquid conductor may no longer touch the patient.

The movable liquid conductor makes it possible in an advantageous way to easily apply the liquid to the patient in the delivery position and simultaneously gives simple protection for the liquid conductor in the standby position.

In an embodiment of the marking apparatus the marking apparatus includes a spring element. The spring element is configured to position the liquid conductor automatically back into the standby position again. For example, the spring element may be connected to the liquid conductor and the body of the marking apparatus, so that the spring element is tensioned in the delivery position.

Automatically includes when the liquid conductor returns to the standby position without the operating element directly bringing about the movement along the axial guide through the effect of a force. The force for the movement may be provided by the spring element. A stop may be actuated by the operating element that prevents retraction by the spring element. A force against the force of the spring element may also be exerted on the liquid conductor via the operating element by the user of the marking apparatus, so that on release of the operating element the liquid conductor is moved by the spring element back into the standby position.

Conversely the spring element may be tensioned in the standby position and moves back automatically on release of the liquid conductor into the delivery position. In this embodiment the marking may be made easier since the actuation of the spring requires less force and thus the precision of the handling is improved.

In an advantageous way the spring element makes possible an automatic return of the liquid conductor from one state into the other and in this way makes the handling of the marking apparatus easier.

One an embodiment, a system made up of the marking apparatus and a liquid reservoir is provided. The liquid reservoir and the marking apparatus are configured to be coupled at the end of the marking opening to the delivery opening in such a way that the liquid conductor receives liquid from the liquid reservoir. For example, the liquid reservoir may have an opening into which the marking apparatus is able to be introduced, so that the delivery opening comes into contact with the liquid and may take up liquid from the liquid reservoir.

In an advantageous way the liquid for application to the patient may be taken up easily and reliably with the liquid reservoir. Over and above this the liquid reservoir may serve as a cap for protection of the liquid conductor from contamination, damage or drying out.

In an embodiment of the marking apparatus the body of the marking apparatus includes a hollow area for accommodating the liquid. The hollow area may accommodate the liquid itself or a container with the liquid. The hollow area makes a closed liquid connection with the delivery opening, so that the liquid may get from the hollow area to the delivery opening. In this case the liquid may get to the delivery opening without losses. For example, the liquid connection may be provided by a small tube between the hollow space or the container and the delivery opening.

The hollow space allows storage of a larger amount of the liquid and makes it possible to mark a plurality of points without having to put the marking apparatus down.

In an embodiment of the marking apparatus the marking apparatus includes a valve. A valve here refers to any facility that makes it possible to influence or to interrupt a flow of the liquid between the hollow space or container controlled by the user via the operating element. The valve is arranged between the hollow area and the delivery opening. For example, the valve, as a stop valve, may interrupt the flow or the connection via the liquid connection between hollow space and delivery opening. A control of the amount of fluid flowing in a plurality of stages may be provided.

The valve makes possible a smooth and supervised control of the delivery of the fluid.

In an embodiment of the marking apparatus the marking apparatus includes a conveying apparatus for the liquid. The conveying apparatus is arranged in the liquid connection between the hollow space and the delivery opening. A conveying apparatus may be any apparatus that, on actuation via the actuation element, actively conveys a predetermined amount of liquid from the hollow space or container. The conveying apparatus may be a mechanical or electrical pump for example, that is actuated or switched on via the actuation element.

In an advantageous way a conveying apparatus makes possible the application of liquids with high viscosity.

In an embodiment of the marking apparatus the actuation element is arranged at one end of the longitudinal body at which the delivery opening is located. In this case the actuation element may be arranged adjacent to the delivery opening. Adjacent to the delivery opening is to be understood as the actuation element being located, for a subdivision of the body into two parts of equal length, along an axis of the greatest extent in the half of the longitudinal body on which the delivery opening is also arranged. The actuation element may be located in a position in which, for a guidance of the instrument in a user's hand as per application, a finger is located for actuation of the actuation element. For example, the marking apparatus may be guided like a pen or ballpoint pen between thumb, first finger and middle finger and in this case the actuation element be located under one of the fingers.

In an advantageous way the arrangement of the actuation element close to the delivery opening makes possible a sensitive guidance in the manner of a pen.

In an embodiment of the marking apparatus the actuation element is arranged in the middle along a longitudinal extent of the longitudinal body. Middle may be not only the precise midpoint on the half of the longitudinal extent but also a location in a middle third of the middle of the longitudinal extent.

In an advantageous way an arrangement in the middle ensures a distance between the operator's hand and the patient, that makes it possible to see the working area and maintains the sterility by avoiding direct contact between hand and patient.

### BRIEF DESCRIPTION OF THE FIGURES

FIG. 1 depicts a schematic diagram of a magnetic resonance tomograph for use with the marking apparatus according to an embodiment.

FIG. 2 depicts a schematic diagram of an embodiment of the marking apparatus according to an embodiment.

FIG. 3 depicts a schematic diagram of an embodiment of the marking apparatus according to an embodiment.

FIG. 4 depicts a schematic diagram of a liquid being taken up by an marking apparatus according to an embodiment.

FIG. 5 depicts a schematic diagram of an embodiment of the marking apparatus according to an embodiment.

FIG. 6 depicts a schematic diagram of an actuation element of an marking apparatus according to an embodiment.

FIG. 7 depicts a schematic diagram of an embodiment of the marking apparatus according to an embodiment.

### DETAILED DESCRIPTION

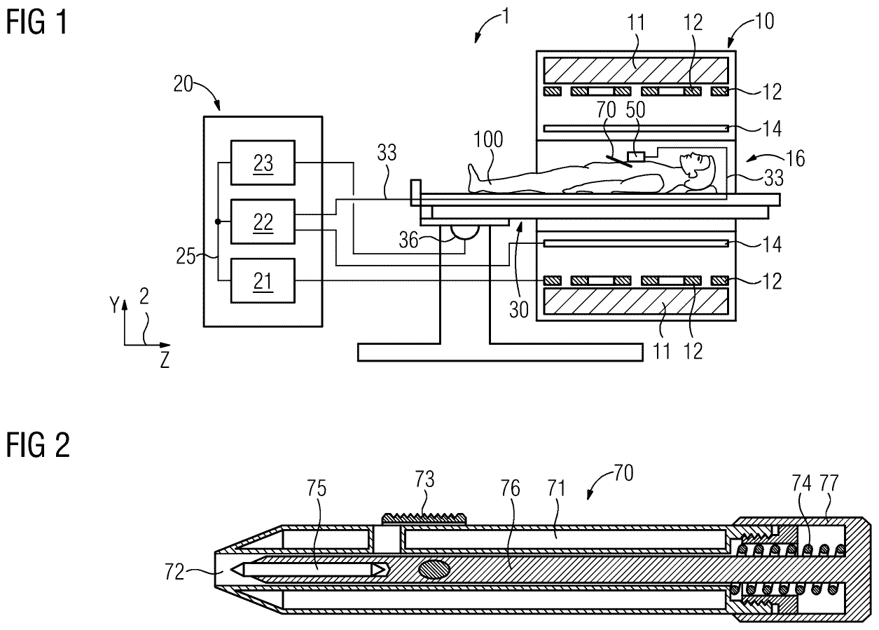

FIG. 1 depicts a schematic diagram of a magnetic resonance tomograph for use with the marking apparatus.

The magnet unit 10 includes a field magnet 11, that creates a static magnetic field B0 for aligning nuclear spins of samples or of the patient 100 in a recording area. The recording area is characterized by an extremely homogeneous static magnetic field B0, wherein the homogeneity for example relates to the magnetic field strength or to its amount. The recording area is almost spherical in shape and is arranged in a patient tunnel 16 that extends in a longitudinal direction 2 through the magnet unit 10.

A patient couch 30 is able to be moved in the patient tunnel 16 by the drive unit 36.

The field magnet 11 may involve a superconducting magnet, that may provide magnetic fields with a magnetic flux density of up to 3 T, in the most recent devices even beyond this. For lower field strengths, however, permanent magnets or electromagnets with normally conducting coils may be used.

Furthermore, the magnet unit 10 includes gradient coils 12, that are configured, for spatial differentiation of the acquired imaging regions in the examination volume, to superimpose variable magnetic fields in three spatial directions onto the magnetic field B0. The gradient coils 12 may be coils made from normally conducting wires, that may create fields orthogonal to one another in the examination volume.

The magnet unit 10 includes a body coil 14, that is configured to radiate a radio frequency signal supplied via a signal line into the examination volume and to receive resonance signals emitted from the patient 100 and output them via a signal line. The term transmit antenna below refers to an antenna via which the radio-frequency signal to excite the nuclear spins is emitted. This may be the body coil 14, but also a local coil 50 with a transmit function.

A control unit 20 supplies the magnet unit 10 with the various signals for the gradient coils 12 and the body coil 14 and evaluates the received signals.

Thus the control unit 20 includes a gradient controller 21, that is configured to supply the gradient coils 12 via supply lines with variable currents that, temporally coordinated, provide the desired gradient fields in the examination volume.

Furthermore, the control unit 20 includes a radio frequency unit 22, that is configured to create a radio-frequency pulse with a predetermined waveform, amplitude and spectral power distribution for exiting a magnetic resonance of the nuclear spins in the patient 100. In this case pulse powers in the region of kilowatts may be achieved. The excitation signals may be emitted via the body coil 14 or also via a local transmit antenna into the patient 100.

A controller 23 communicates via a signal bus 25 with the gradient controller 21 and the radio frequency unit 22.

Arranged on the patient 100 is a local coil 50, that is linked via a connecting line 33 to the radio frequency unit 22 and its receiver. The body coil 14 may be a receive antenna.

By a marking apparatus 70 an optically visible marking and/or a marking able to be shown in an image in the magnetic resonance tomograph 1 is applied to a patient 100 by an optically visible liquid and/or a liquid active for magnetic resonance.

FIG. 2 depicts a schematic diagram of a form of embodiment of the marking apparatus 70. The marking apparatus includes a longitudinal body 71 or housing. The body 71 may be configured in the shape of a cylinder for example or in another geometrical shape, that is to be guided ergonomically in the hand of a user. The body 71, in an axial direction of extent, includes a dimension, also referred to as length, that is a multiple of the dimensions in directions at right angles to this axial direction of extent, that is also referred to as width. For example the length is three times, five times or ten times greater than the width. The body 71 may be configured in one piece or may also be composed of a number of parts.

Arranged at one end of the body 71 along the axial extent is a delivery opening, at which the marking apparatus 70 may deliver a liquid active for magnetic resonance and/or optically visible, as described below. The longitudinal body 71 may include a reduced breadth or width at the delivery opening 72 and may be formed into a conical tip for example, so that a view of the delivery opening or the point on the patient 100 to be marked is possible when the marking apparatus 70 is being held in accordance with its intended use.

An actuation element 73 is arranged laterally in the form of embodiment shown as a slider on the longitudinal body 71. In the embodiment of FIG. 2 it is located in the immediate vicinity of the delivery opening 72 and is able to be pushed in the direction of the axial extent. However a tangential, radial or in a mixed form such as an actuation element 73 able to be moved on helical track relative to the body 71 may be used, as is also shown partly below for other embodiments.

A liquid conductor 75 is arranged in the body 71 coaxial to the delivery opening 72 and is able to be moved in the axial longitudinal direction relative to the body 71. The liquid conductor 75 is force coupled to the actuation element 73, for example via a slot and a tab that engages in it, so that with an actuation of the actuation element 73 the liquid conductor 75 is moved in the axial longitudinal direction through to the delivery opening 72 and is moved through said opening, so that the liquid conductor 75 projects beyond the body 71 and may deliver the liquid onto the patient 100.

The liquid conductor 75 includes a liquid connection to the liquid reservoir 76. For example, the liquid conductor 76 may be a capillary conductor such as a fiber bundle, that is arranged in the liquid reservoir 76 at its end opposite to the delivery opening 72. In this case the liquid reservoir 76, on actuation of the actuation element 73, may be moved jointly with the liquid conductor 75. However a flexible liquid connection such as a hose may be arranged between liquid reservoir 76 and the liquid conductor 75, so that the liquid reservoir 76, on actuation of the actuation element, remains arranged in a fixed relative position in the body 71. For liquids with high viscosity the liquid conductor 75 itself may likewise be configured as a hose or tube.

In an embodiment the marking apparatus 70 furthermore includes a spring element 74, that is connected for transmission of a force to the body 71 and the actuation element 73. The spring element 74 in FIG. 2 is configured to move the liquid conductor 75 pulled back from the delivery position into the standby position in the delivery opening if an opposing force is not being applied via the actuation element 73. In the embodiment of FIG. 2 the spring element 74 is arranged at the end of the longitudinal body 71 opposite to the delivery opening. The spring element 74 is fixed via an end cap 76 relative to the body 71 and exerts a force on the actuation element and the liquid conductor 75 via the liquid reservoir 76. The spring element 74 may be arranged in the end of the body 71 adjacent to the delivery opening 72, for example when the actuation element 73 is at a greater distance from the delivery opening 72. Likewise the liquid conductor 75 may be moved solely via the actuation element 73 without a spring element 74.

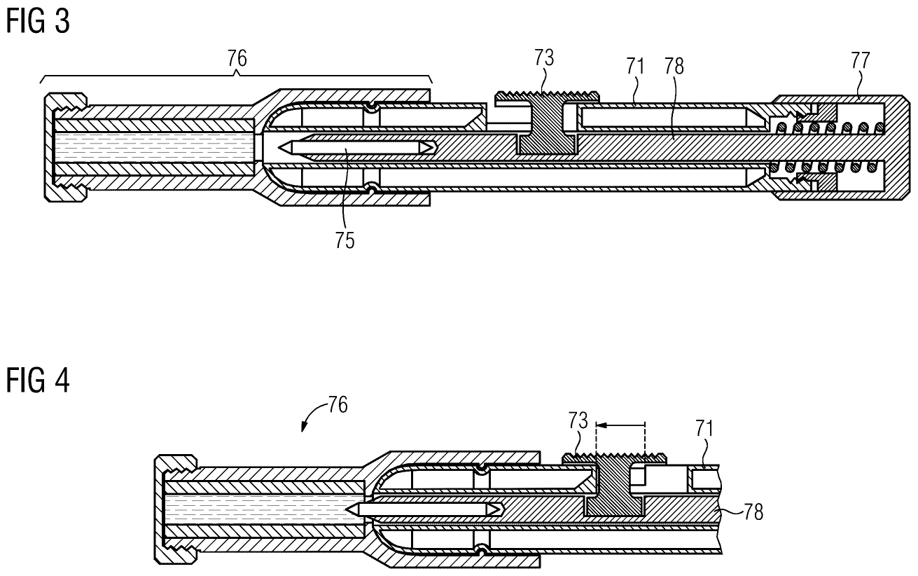

FIG. 3 presents an embodiment of the marking apparatus, in which the liquid reservoir 76 is not arranged in the body 71 but as an external container, that is placed or pushed over the delivery opening 72. The force coupling between the spring element 74 on the one hand and the liquid conductor 75 and also the actuation element 73 on the other hand is provided by a driver 78 in the body 71.

The liquid reservoir 76 is pushed like a cap over the delivery opening 72. The liquid is kept in the inside of the liquid reservoir 76. For liquids with lower viscosity the liquid may be stored for example in a fleece in the inside of the liquid reservoir 76. For liquids with high viscosity, such as for example a gel, this may also be kept directly in the liquid reservoir 76 without a carrier medium. To enable the liquid reservoir 76 to be pushed over the body 71, in the embodiment of FIG. 3 and FIG. 4 the actuation element 73 is displaced more to the middle on the body 71. The liquid reservoir 76 and the body 71 may include corresponding retaining structures such as a slot and a tab, that through an elasticity of the liquid reservoir 76, fix this in the pushed-on state on the body 71. The liquid reservoir 76 may include a shell or cap and a liquid container arranged therein.

FIG. 4 depicts a schematic diagram of the take-up of a liquid by a marking apparatus 70.

As already explained for delivery of the liquid, the liquid conductor 75 is moved into the delivery position by the actuation element 73, in which it projects out of the delivery opening 72. With the liquid reservoir 76 pushed on however the liquid conductor 75 reaches into the liquid kept in the liquid reservoir 76. In this position the liquid is taken up by the liquid conductor 75, for example for liquids with low viscosity by capillary forces or with high viscosity by adhesion forces.

The embodiment of FIG. 5 differs above all from the embodiment of FIG. 3 in that the spring element 74 in the non-tensioned state does not pull the liquid conductor 75 via the driver 78 back into the standby position within the body 71 but pushes it into the delivery position. For this the spring element 74 must be pretensioned by the user, for example by pulling back the closing cap 77. When this is done the driver 78 and the liquid conductor 75 are likewise pulled back.

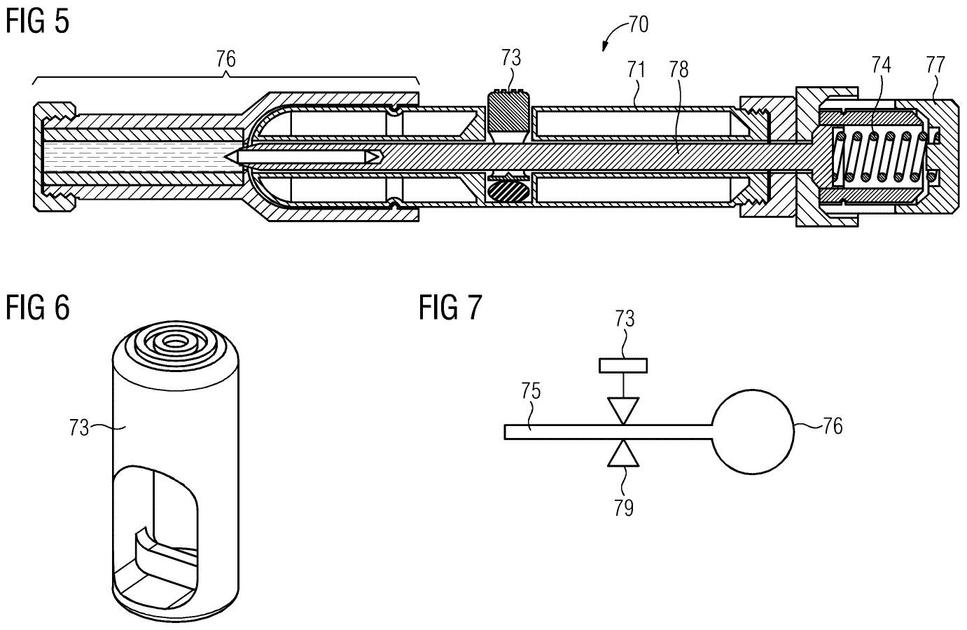

The actuation element 73 together with the driver 78 includes corresponding latching elements engaging with each other, that until the actuation of the actuation element 73 fix the fluid conductor 75, the driver 78 and the spring element 74 in the tensioned position. For example, the driver 78 may have a slot into which a tab of the actuation element 73 engages and through a further spring element holds the actuation element 73 in this position. FIG. 6 depicts a corresponding actuation element 73. With the actuation of the actuation element 73 this lock is released and the spring element 74 conveys the liquid conductor 75 into the delivery position, in which a marking may be applied to the patient 100 or, with a pushed-on liquid reservoir 76, liquid may be taken up from the liquid conductor 75.

FIG. 7 depicts schematically the liquid-guiding elements of an embodiment of the marking apparatus 70.

This embodiment differs in that the liquid is actively conveyed through the liquid conductor 75. In this case the liquid reservoir 76 may store the liquid under pressure. For example, the liquid reservoir 75 may additionally have a compression gas. A cylinder-piston combination may be used, in which a spring exerts a pressure on the piston. The actuation element 73 in this embodiment amounts to a valve 79, that makes a liquid connection between the liquid conductor 75 and the liquid reservoir 76.

An embodiment in which the actuation element 76 directly actuates a mechanical pump for the liquid or actuates an electric pump may be provided.

It is to be understood that the elements and features recited in the appended claims may be combined in different ways to produce new claims that likewise fall within the scope of the present disclosure. Thus, whereas the dependent claims appended below depend from only a single independent or dependent claim, it is to be understood that the dependent claims may, alternatively, be made to depend in the alternative from any preceding or following claim, whether independent or dependent, and that such new combinations are to be understood as forming a part of the present specification.

While the present disclosure has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A marking apparatus for applying a liquid that is optically visible, visible to magnetic resonance, or optically visible and visible to magnetic resonance to a patient in a magnetic resonance tomograph, the marking apparatus comprising:
a longitudinal body with material visible to magnetic resonance and a delivery opening at an end of the longitudinal body; and
an actuation element that is arranged laterally on the longitudinal body of the marking apparatus and controls a delivery of the liquid to the delivery opening.

2. The marking apparatus of claim 1, further comprising:
a liquid conductor that is arranged movably in the delivery opening and by the actuation element from a delivery position, in which the liquid conductor is configured to deliver the liquid to the delivery opening onto a surface of the patient located there, into a standby position, in which no delivery of the liquid at the surface of the patient located at the delivery opening may take place.

3. The marking apparatus of claim 2, further comprising:
a spring element that is configured to position the liquid conductor automatically back in the standby position.

4. The marking apparatus of claim 2, further comprising:
a spring element that is configured to position the liquid conductor automatically in the delivery position.

5. The marking apparatus of claim 2, wherein a body of the marking apparatus includes a liquid reservoir for accommodating the liquid, wherein the liquid reservoir and the marking apparatus are configured to be coupled to the delivery opening at the end of the longitudinal body in such a way that the liquid conductor takes up liquid from the liquid reservoir.

6. The marking apparatus of claim 5, further comprising:
a valve that is arranged between the liquid reservoir and the delivery opening and is configured to influence a flow of the liquid between the liquid reservoir and the delivery opening depending on an actuation of the actuation element.

7. The marking apparatus of claim 5, further comprising:
a conveying apparatus for the liquid, wherein the conveying apparatus is configured, with an actuation of the actuation element to convey liquid from the liquid reservoir to the delivery opening.

8. The marking apparatus of claim 1, wherein the actuation element is arranged at one end of the longitudinal body at which the delivery opening is located.

9. The marking apparatus of claim 1, wherein the actuation element is arranged in a middle along a longitudinal extent of the longitudinal body.

10. A system comprising:
a marking apparatus comprising:
a longitudinal body with material visible to magnetic resonance and a delivery opening at an end of the longitudinal body; and
an actuation element that is arranged laterally on the longitudinal body of the marking apparatus and controls a delivery of a liquid to the delivery opening; and
a liquid reservoir,
wherein the liquid reservoir and the marking apparatus are configured to be coupled to the delivery opening at the end of the longitudinal body in such a way that a liquid conductor takes up liquid from the liquid reservoir.

11. The system of claim 10, wherein the marking apparatus further comprises:
a liquid conductor that is arranged movably in the delivery opening and by the actuation element from a delivery position, in which the liquid conductor is configured to deliver the liquid to the delivery opening onto a surface of a patient located there, into a standby position, in which no delivery of the liquid at the surface of the patient located at the delivery opening may take place.

12. The system of claim 11, wherein the marking apparatus further comprises: further comprising:
a spring element that is configured to position the liquid conductor automatically back in the standby position.

13. The system of claim 11, wherein the marking apparatus further comprises:
a spring element that is configured to position the liquid conductor automatically in the delivery position.

14. The system of claim 10, wherein the marking apparatus further comprises:
a valve that is arranged between the liquid reservoir and the delivery opening and is configured to influence a flow of the liquid between the liquid reservoir and the delivery opening depending on an actuation of the actuation element.

15. The system of claim 10, wherein the marking apparatus further comprises:
a conveying apparatus for the liquid, wherein the conveying apparatus is configured, with an actuation of the actuation element to convey liquid from the liquid reservoir to the delivery opening.

16. The system of claim 10, wherein the actuation element is arranged at one end of the longitudinal body at which the delivery opening is located.

17. The system of claim 10, wherein the actuation element is arranged in a middle along a longitudinal extent of the longitudinal body.
